(19) United States
(12) Patent Application Publication
(10) Pub. No.: US 2007/0243103 A1
(43) Pub. Date: Oct. 18, 2007

(54) Disinfectant system

(76) Inventors: Helrik Bobke, Hannover (DE); Ralf Wolbers, Schuettorf (DE)

Correspondence Address: GREENBLUM & BERNSTEIN, P.L.C., 1950 ROLAND CLARKE PLACE, RESTON, VA, 20191, US

(21) Appl. No.: 11/407,042

(22) Filed: Apr. 20, 2006

(30) Foreign Application Priority Data
Aug. 11, 2005 (DE) 10 2005 038 320.3

(51) Int. Cl. A61L 2/20

(52) U.S. Cl. 422/28; 206/219; 422/305; 422/37

(57) ABSTRACT

Portable container for producing a disinfectant. The container includes an exterior wall made of a gas-permeable material and two or more chambers arranged adjacent to one another structured and arranged to separately hold ready-made solutions or dry substances for the production of a gaseous disinfectant. A wall arranged between the two or more chambers is impermeable to substances and is capable of being made permeable manually prior to the use of the disinfectant. This Abstract is not intended to define the invention disclosed in the specification, nor intended to limit the scope of the invention in any way.

### CROSS REFERENCE TO RELATED APPLICATIONS

This application claims priority under 35 U.S.C. §119 to German Application Serial No. 10 2005 038 320.3, filed on Aug. 11, 2005, which application claims priority to German Application Serial No. 10 2004 039 367.2, filed on Aug. 12, 2004.

### BACKGROUND OF THE INVENTION

1. Field of the Invention

The invention relates to a portable container for disinfecting, e.g., storage spaces or liquids or installations. This way, a disinfection can be carried out that can be applied by staff in a simple, safe and time-efficient manner.

2. Background of the Invention

Disinfectants are frequently used industrially, and also in private households. In particular, in the case of private users, the use of prefabricated, ready-made disinfectants in the form of liquids or sprays is currently known. Such liquids or sprays, however, respectively have to be taken from a storage container and applied for disinfection to the areas to be disinfected.

Chlorine dioxide has been used since the 1950s for disinfection drinking water. The chlorine dioxide is added to the water to be purified in an aqueous solution. In this respect, however, there are several problems. The reagents must be mixed several hours before use and are then, in turn, admixed to the water. The reagents are also problematic in terms of handling. If the disinfectant is used in a throughput arrangement (as occurs in an installation), expensive dosing devices are required in order to, on the one hand, prevent waste of the disinfectant, and, on the other hand, guarantee reliable disinfection.

### SUMMARY OF THE INVENTION

The invention relates to a portable container for producing a disinfectant.

The invention also provides a portable container for disinfecting, e.g., storage spaces or liquids that renders superfluous an open mixing, removing of the disinfectant from the container and a manual application of the disinfectant to the area to be disinfected.

The invention also provides a container composed of two or more chambers adjacent to one another for separately holding ready-made solutions and/or dry substances for the production of a gaseous disinfectant. The wall of the chambers adjacent to one another can be made of impermeable material which can, however, be made manually permeable prior to being used.

The exterior wall of the container can be made of a gas-permeable material, and there is no need for a separate outlet. The basic substances for the production of a gaseous disinfectant can be stored separately in the portable container. When the disinfectant is to be used, the substances separated by an interior wall can be mixed with one another with one manipulation by destroying the interior wall, so that the basic substances react to form a gaseous disinfectant that escapes into the environment through the gas-permeable container wall.

The invention also provides a portable container for producing a disinfectant. The container may include an exterior wall made of a gas-permeable material, and two or more chambers arranged adjacent to one another structured and arranged to separately hold ready-made solutions or dry substances for the production of a gaseous disinfectant. A wall arranged between the two or more chambers can be impermeable to substances and is capable of being made permeable manually prior to the use of the disinfectant.

The exterior wall of the container may have no separate outlet. The two or more chambers may comprise a first chamber containing a chlorite solution and a second chamber containing an oxidant. The oxidant may have a pH value of less than 5 and the chlorite solution may have a pH value of more than 9. The wall may comprise a joint wall that separates the two or more chambers. The two or more chambers may comprise a first chamber arranged within a second chamber. The first chamber may be arranged completely within the second chamber filled with substance, and the second chamber may completely adjoins a container wall. The container may have the form of an oval capsule. The container may have the form of a tube. The container may comprise an exterior surface which includes an attachment device. The attachment device may comprises a hook. The attachment device may comprise an adhesive strip. The container may be made of a more or less gas-permeable sheet, which ensures that an interior pressure of the container equals an exterior pressure, whereby the container is capable of diffusing disinfecting gas. The container may be made of polyethylene (PE).

The invention also provides for a method of using the container described herein wherein the method comprises disinfecting a storage space or a cold store.

The invention also provides for a method of disinfecting with the container described herein wherein the method comprises disinfecting a storage space or a cold store stored with foodstuffs.

The invention also provides for a method of using the container described herein wherein the method comprises disinfecting an unpressurized or a pressurized liquid-bearing system and liquids contained therein.

The invention also provides for a method of using the container described herein wherein the method comprises disinfecting an unpressurized or a pressurized liquid-bearing installation and liquids contained therein.

The invention also provides for a portable disinfecting container comprising an exterior wall made of a gas-permeable material, at least two chambers arranged adjacent to one another structured and arranged to separately hold different substances, which when mixed with one another, produce a gaseous disinfectant, and a wall separating the at least two or more chambers being impermeable to substances and being capable of being made permeable.

The at least two chambers may comprise a first chamber arranged within a second chamber.

The invention also provides for a method of producing a gaseous disinfectant using a portable disinfecting container comprising an exterior wall made of a gas-permeable material, at least two chambers arranged adjacent to one another structured and arranged to separately hold substances, which when mixed with one another, produce a gaseous disinfectant, and a wall separating the two or more chambers that is impermeable to the substances, wherein the method comprises one of causing the substances to mix with one another by manually causing the wall to become permeable to the substances and causing the substances to mix with one another by destroying the wall so that the wall becomes permeable to the substances.

### DETAILED DESCRIPTION OF THE INVENTION

The invention provides a portable container for disinfecting, e.g., storage spaces or liquids or installations. As a result, disinfection can be carried out by staff in a simple, safe and time-efficient manner. In particular, manipulations that involve open handling of reagents, removal from the container and manual application of the disinfectant to the area to be disinfected can be avoided as far as possible and need no longer be necessary.

To this end, the invention provides for creating a disinfecting system in the form of a container composed of two or more chambers adjacent to one another. The chambers are suitable for separately holding ready-made solutions and/or dry substances for the production of a gaseous disinfectant and are filled with these substances. Additionally, the separating wall of the chambers adjacent to one another is made of a material impermeable to the substances. The separating wall can, however, manually be made permeable prior to the use of the disinfectant. The exterior wall of the container is made of a material impermeable to the substances and permeable to the gaseous disinfectant.

The invention also provides that the basic substances for the production of a gaseous disinfectant are stored separately in the portable container. When the disinfectant is to be used, the substances that are separated from one another by a wall (referred to as an interior wall or the separating wall) can be mixed with one another, so that the basic substances react to form the gaseous disinfectant.

The substances can be, e.g., firstly, a starting material and, secondly, an activator, whereby the substances do not have to be in the same state of aggregation. Purely by way of example, the substances can thus each be present as a liquid, or as a solid and a liquid substance In addition, although the use of two substances is described within the scope of the present specification, the invention also contemplates using more than two substances. However, the use of two substances has the advantage that a reliable mixing can be assured in a particularly simply manner. Furthermore, it is not necessary to keep in mind whether two particular substances must first react with one another before this mixture reacts with a third substance, so that with the use of two substances the desired mixing result, i.e., the disinfectant to be produced, can be ensured with very high reliability.

This mixing of the substances can preferably be carried out by way of a single manipulation of the container, whereby the interior separating wall is destroyed, so that a simple and trouble-free handling of the disinfection device, i.e., the container, is possible without using tools.

According to the invention, the disinfectant does not have to be discharged into the environment via a separate outlet that needs to be opened, as is known. Instead, with the container according to the invention, the gaseous disinfectant escapes into the environment solely through the gas-permeable container wall. It can thus advantageously be provided that the exterior container wall is embodied without an otherwise customary filling or outlet opening. This results in a handling of the container that is as safe as possible for the user, since no closing device can be accidentially opened. In particular, for the application in private households where—in contrast to industrial applications of the disinfectant—no technical staff is trained to handle this disinfectant, the disinfection system according to the invention can be handled by laypersons, which results in a very high degree of safety.

The production of the disinfection system can easily be carried out industrially by filling the container, e.g., made of plastic and open at one point, and welding together its exterior wall after filling. If the exterior wall is embodied as a bag, a full-perimeter seam can be welded, e.g., in a manner known per se to close the bag. If the exterior wall is embodied as a tube, a cap can be welded onto one end of the tube, e.g., in a manner known per se to close the tube.

When the basic substances have been mixed, the container can be placed into the area to be disinfected or into the liquid to be disinfected or it can be installed in the installation for disinfecting at a suitable location. On account of the independent degassing, which occurs via either part of the container surface or via the entire container surface, the use of the disinfectant is simple and can save time. The application to the area to be disinfected can be less labor intensive because the disinfecting gas spreads in a defined area or a liquid and there disinfects automatically, i.e., “independently.”

The present invention also makes it possible to activate the substances in the container without touching the reagents and to use them directly. The activation process can take place inside the container without being disturbed by the medium to be disinfected—such as, e.g., air or water. After the activation has occurred, the disinfectant, e.g., chlorine dioxide, starts to diffuse through the container wall into the medium.

According to another aspect of the invention, there is no need for any contact between the user and the reagents or the finished solution. The inhalation of the ready-mixed disinfectant, e.g., the chlorine dioxide, produced from the substances, can also be prevented as far as possible, by ensuring that the manipulation of the container is of short duration, when the interior separating wall is made permeable to the substances, e.g. when it is opened or destroyed. The container can then be placed into the area to be disinfected, and the user can close-off the area or get away from the area. According to the invention, sufficient time before the disinfectant is formed from the substances and has escaped through the exterior wall of the container, should exist so that the user does not inhale the disinfectant.

In the field of so-called “point-of-use reverse osmosis systems” or “small-amount reverse osmosis installations,” no effective and at the same time user-friendly disinfectant is currently known on the market that can disinfect the entire installation including the reverse osmosis membrane. In this respect, hydrogen peroxide is problematic in terms of handling and easily affects the sensitive membranes. Although peracetic acid is better compatible with the membranes, it cannot be shipped in a normal manner, as it can be transported only in ventilated containers. In addition, it is highly caustic and causes severe skin irritations. Other agents can be used only on respectively one side, i.e., only before the membrane and/or only after the membrane, since they cannot penetrate it, and thus have to be dosed correctly and introduced into the installation on both sides of the membrane, if necessary. The really critical part, i.e., the membrane on the high-purity-water side or permeate side, is thereby generally not reached, as it is difficult to access.

Through the characteristic of the disinfectant used according to the invention, in particular, having the form of a gas, e.g., chlorine dioxide, which can penetrate plastics, it is rendered possible to, e.g., disinfect such small reverse osmosis installations from the pre-filter through the reverse osmosis membrane right into the storage tank. The individual reagents are, as a result, not released in the system. Instead, only the disinfecting gas escapes after a reaction time and this gas is distributed, e.g., as free chlorine dioxide, in the system through the normal operation of the installation. If the reverse osmosis installation stagnates, i.e., if there is no water flow, the disinfecting gas greatly enriches the water quantity located around the place of use of the disinfection system.

The amount released per unit time and the duration of effectiveness can be controlled depending on the size and material of the container exterior. A flat sheet bag with approximately 50 ml basis material and a small bag with an activator liquid floating in this bag is sufficient to disinfect a commercially available household installation. When the activator bag located within has been crushed—with the exterior container remaining undamaged—this package can be accommodated, e.g., together with a commercial filter, referred to as a “filter cartridge,” in the pre-filter or in the membrane chamber of the reverse osmosis installation, where it does not interfere in the longer run. Over several days disinfectant, e.g., chlorine dioxide, can be released in the installation, and this disinfectant then diffuses through the membrane and is still detectable at a large distance, even in the storage tank of the installation, and has its disinfecting effect there. A service technicians or even a technically untrained end user, can thus insert such a disinfection package into the system during a filter change, which is provided at regular intervals anyway, and thereby disinfect the installation. Moreover, this can occur without having to take particular safety precautions. The bag can remain in the installation until the next service.

In most small reverse osmosis installations, an activated-carbon final filter is provided between the permeate storage tank and the permeate withdrawal site, e.g., a spigot. If such an activated-carbon final filter is replaced as well during the filter change mentioned, so that a fresh, unused activated-carbon final filter is available in the installation at the beginning of the disinfection, then this activated-carbon final filter is capable of almost completely filtering out the chlorine dioxide effective in small amounts in the storage tank. As such, the user can use the product water even during the disinfection phase of the installation.

1-12. (canceled)

13. A portable container for producing a disinfectant, comprising:
an exterior wall made of a gas-permeable material;
two or more chambers arranged adjacent to one another structured and arranged to separately hold ready-made solutions or dry substances for the production of a gaseous disinfectant; and
a wall arranged between the two or more chambers being impermeable to substances and being capable of being made permeable manually prior to the use of the disinfectant.

14. The container of claim 13, wherein the exterior wall of the container has no separate outlet.

15. The container of claim 13, wherein the two or more chambers comprises a first chamber containing a chlorite solution and a second chamber containing an oxidant.

16. The container of claim 15, wherein the oxidant has a pH value of less than 5 and the chlorite solution has a pH value of more than 9.

17. The container of claim 13, wherein the wall comprises a joint wall that separates the two or more chambers.

18. The container of claim 13, wherein the two or more chambers comprises a first chamber arranged within a second chamber.

19. The container of claim 18, wherein the first chamber is arranged completely within the second chamber filled with substance, and the second chamber completely adjoins a container wall.

20. The container of claim 13, wherein the container has the form of an oval capsule.

21. The container of claim 13, wherein the container has the form of a tube.

22. The container of claim 13, wherein the container comprises an exterior surface which includes an attachment device.

23. The container of claim 22, wherein the attachment device comprises a hook.

24. The container of claim 22, wherein the attachment device comprises an adhesive strip.

25. The container of claim 13, wherein the exterior wall is made of a gas-permeable sheet, which ensures that an interior pressure of the container equals an exterior pressure, whereby the container is capable of diffusing disinfecting gas.

26. The container of claim 13, wherein the exterior wall is made of polyethylene (PE).

27. A method of disinfecting with the container of claim 13, the method comprising:
disinfecting a storage space or a cold store.

28. A method of using the container of claim 13, the method comprising:
disinfecting a storage space or a cold store stored with foodstuffs.

29. A method of using the container of claim 13, the method comprising:
disinfecting an unpressurized or a pressurized liquid-bearing system and liquids contained therein.

30. A method of using the container of claim 13, the method comprising:
disinfecting an unpressurized or a pressurized liquid-bearing installation and liquids contained therein.

31. A portable disinfecting container, comprising:
an exterior wall made of a gas-permeable material;
at least two chambers arranged adjacent to one another structured and arranged to separately hold different substances, which when mixed with one another, produce a gaseous disinfectant; and
a wall separating the at least two or more chambers being impermeable to substances and being capable of being made permeable.

32. The container of claim 31, wherein the at least two chambers comprises a first chamber arranged within a second chamber.

33. A method of producing a gaseous disinfectant using a portable disinfecting container comprising an exterior wall made of a gas-permeable material, at least two chambers arranged adjacent to one another structured and arranged to separately hold substances, which when mixed with one another, produce a gaseous disinfectant, and a wall separating the two or more chambers that is impermeable to the substances, the method comprising one of:
causing the substances to mix with one another by manually causing the wall to become permeable to the substances; and
causing the substances to mix with one another by destroying the wall so that the wall becomes permeable to the substances.
